(19) United States
(12) Patent Application Publication
(10) Pub. No.: US 2002/0121274 A1
(43) Pub. Date: Sep. 5, 2002

(54) Laminated electroformed aperture plate

(75) Inventors: Scott Borland, San Mateo, CA (US); Jonathan Martin, Sunnyvale, CA (US); Scott Semans, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AEROGEN, INC.

(21) Appl. No.: 09/970,335

(22) Filed: Oct. 2, 2001

Related U.S. Application Data
(63) Continuation-in-part of application No. 09/313,914, filed on May 18, 1999.
Application No. 09/313,914 is a continuation-in-part of application No. 09/149,426, filed on Sep. 8, 1998, now Pat. No. 6,205,999.
Application No. 09/149,426 is a continuation of application No. 09/095,737, filed on Jun. 11, 1998, now Pat. No. 6,014,970.
Application No. 09/149,426 is a continuation of application No. 08/417,311, filed on Apr. 5, 1995, now Pat. No. 5,938,117.

(51) Int. Cl. A61M 11/00; A61M 15/00

(52) U.S. Cl. 128/200.16; 128/200.14; 128/203.12

(57) ABSTRACT

An aperture plate comprises a plate body having a top surface, a bottom surface, and tapered walls that form a plurality of apertures that taper from the bottom surface to the top surface wherein the plate body comprises a base material and a corrosion resistive material plating at least the tapered walls. The aperture plate can be produced by electroplating a mandrel wherein the mandrel is placed within a solution containing a material that is to be deposited onto the mandrel. Electrical current is applied to the mandrel to form the aperture plate on the mandrel. The process can be repeated so as to deposit a second layer, such as a corrosion resistive material, over a base layer material. In such fashion, a laminated electroformed aperture plate can be produced.

### CROSS-REFERENCES TO RELATED APPLICATIONS

This application is a continuation in part application of U.S. patent application Ser. No. 09/313,914, filed May 18, 1999, which is a continuation in part application of U.S. patent application Ser. No. 09/149,426, filed Sep. 8, 1998, now U.S. Pat. No. 6,205,999, which is a continuation application of U.S. patent application Ser. No. 09/095,737, filed Jun. 11, 1998 now U.S. Pat. No. 6,014,970 and a continuation in part application of U.S. patent application Ser. No. 08/417,311, filed Apr. 5, 1995, now U.S. Pat. No. 5,938,117, the complete disclosures of which are herein incorporated by reference.

### BACKGROUND OF THE INVENTION

This invention relates generally to the field of liquid dispensing, and in particular to the aerosolizing of fine liquid droplets. More specifically, the invention relates to the formation and use of aperture plates employed to produce such fine liquid droplets.

A great need exists for the production of fine liquid droplets. For example, fine liquid droplets are used for drug delivery, insecticide delivery, deodorization, paint applications, fuel injectors, and the like. In many medical applications, it may be desirable to produce liquid droplets that are sized small enough to insure that the inhaled drug reaches the deep lung.

U.S. Pat. Nos. 5,164,740; 5,586,550; and 5,758,637, the complete disclosures of which are herein incorporated by reference, describe exemplary devices for producing fine liquid droplets. These patents describe the use of aperture plates having tapered apertures to which a liquid is supplied. The aperture plates are then vibrated so that liquid entering the larger opening of each aperture is dispensed through the small opening of each aperture to produce the liquid droplets. Such devices have proven to be tremendously successful in producing liquid droplets.

Co-pending U.S. patent application Ser. No. 09/392,180, entitled “Improved Aperture Plate And Method For Its Construction And Use,” filed on Sep. 8, 1999 and (U.S. Pat. No. 6,235,177) herein incorporated by reference, describes various techniques for forming aperture plates. The embodiments of the present invention provide alternative techniques for constructing aperture plates. Such techniques may be used to gain cost savings, processability, and performance, among other features.

The invention provides for the construction and use of other aperture plates that are effective in producing fine liquid droplets at a relatively fast rate. As such, it is anticipated that the invention will find even greater use in many applications requiring the use of fine liquid droplets.

### SUMMARY OF THE INVENTION

The invention provides exemplary systems, apparatus, and methods for aerosolizing a solution. One embodiment of the invention provides an aperture plate having a plate body with a top surface, a bottom surface, and tapered walls that form multiple apertures which taper from the bottom surface toward the top surface. The plate body can be comprised of a base material and a corrosion resistive material which plates at least the tapered walls. In this way, a relatively inexpensive material may be used as the base material to reduce the cost of the aperture plate.

The base material can be electroformed into a first layer and a corrosion resistive material can be electroformed into a second layer. For example, the base material can be made of nickel, while the corrosion resistive material can be made of palladium nickel. Furthermore, both the bottom surface of the aperture plate and the tapered walls can be plated with the corrosion resistive material.

In another embodiment, a portion of the aperture plate body can be configured so as to be dome shaped in geometry. However, other shapes are also possible.

The aperture plate can be back plated with the corrosion resistive material so as to deposit the corrosion resistive material on the top surface of the aperture plate in another embodiment of the invention.

In yet another embodiment of the invention, a method of aerosolizing a liquid utilizes an aperture plate having a plate body with a top surface, a bottom surface, and tapered walls that form multiple apertures. The apertures taper from the bottom surface toward the top surface. The plate body is comprised of a base material and a corrosion resistive material plating the tapered walls. A liquid is supplied to the bottom surface and the aperture plate is vibrated to eject the liquid droplets through the apertures.

In another embodiment of the invention a method for forming an aperture plate is provided by providing a mandrel having a mandrel body with a conductive surface and multiple non-conductive islands on the conductive surface. The mandrel is placed into a solution containing a base material and electrical energy is applied to the mandrel to deposit the base material on the mandrel. Conveniently, the base material may be a relatively inexpensive material. An aperture plate is formed having a top surface adjacent the mandrel, a bottom surface and a plurality of tapered walls that define tapered apertures. The mandrel is placed in a solution containing a corrosion resistive material and electrical energy can be applied to the mandrel to deposit the corrosion resistive material onto the bottom surface and/or the tapered walls. In this way, corrosion resistance is provided to areas of the aperture plate which contacts corrosive liquids.

In yet another embodiment of the invention an intermediate step can be utilized to facilitate bonding of the corrosion resistive material to the base material. For example, the base material can be exposed to a chemical so as to facilitate bonding of the corrosion resistive material to the base material.

In another embodiment of the invention the aperture plate can be removed from the mandrel and the top surface of the aperture plate can be back-plated. In one embodiment of the invention the aperture plate can be heat treated so as to alter the properties of the materials, e.g., the corrosion resistive materials, deposited as part of the aperture plate.

### BRIEF DESCRIPTION OF THE DRAWINGS

FIG. 1 is a side view of an aperture plate under one embodiment of the invention.

FIG. 2 is a cross-sectional side view of a portion of the aperture plate of FIG. 1.

FIG. 3 is a partial cross-sectional side view of an aperture plate under one embodiment of the invention.

FIG. 3A is a horizontal cross-sectional view of the aperture plate of FIG. 3 taken along lines A-A.

FIG. 4 is a partial cross-sectional side view of an aperture plate under another embodiment of the invention.

FIG. 4A is a horizontal cross-sectional view of the aperture plate shown in FIG. 4 taken along lines A-A.

FIG. 5 is a top perspective view of one embodiment of a mandrel having nonconductive islands for producing an aperture plate in an electroforming process according to one embodiment of the invention.

FIG. 6 is a flow chart illustrating an embodiment of the invention for forming an aperture plate.

FIG. 7 is a flow chart illustrating a method for aerosolizing a liquid.

### DETAILED DESCRIPTION OF THE INVENTION

The embodiments of the invention provide exemplary aperture plates and methods for their construction and use. The aperture plates of the invention are constructed of a relatively thin plate that may be formed into a desired shape and may include a plurality of apertures that are employed to produce fine liquid droplets when the aperture plate is vibrated. Techniques for vibrating such aperture plates are described generally in U.S. Pat. Nos. 5,164,740; 5,586,550; and 5,758,637, previously incorporated herein by reference. The aperture plates are constructed to permit the production of relatively small liquid droplets at a relatively fast rate. For example, the aperture plates of the invention may be employed to produce liquid droplets having a size in the range from about 2 microns to about 10 microns, and more typically between about 2 microns to about 5 microns. In some cases, the aperture plates may be employed to produce a spray that is useful in pulmonary drug delivery procedures. As such, the sprays produced by the aperture plates may have a respirable fraction that is greater than about 70%, preferably more than about 80%, and most preferably more than about 90% as described in U.S. Pat. No. 5,758,637, previously incorporated by reference.

In some embodiments, such fine liquid droplets may be produced at a rate in the range from about 4 microliters per second to about 30 microliters per second per 1000 apertures. In this way, aperture plates may be constructed to have multiple apertures that are sufficient to produce aerosolized volumes that are in the range from about 4 microliters to about 30 microliters, within a time that is less than about one second. Such a rate of production is particularly useful for pulmonary drug delivery applications where a desired dosage is aerosolized at a rate sufficient to permit the aerosolized medicament to be directly inhaled. In this way, a capture chamber is not needed to capture the liquid droplets until the specified dosage has been produced. In this manner, the aperture plates may be included within aerosolizers, nebulizers, or inhalers that do not utilize elaborate capture chambers.

As just described, the invention may be employed to deliver a wide variety of drugs to the respiratory system. For example, the invention may be utilized to deliver drugs having potent therapeutic agents, such as hormones, peptides, and other drugs requiring precise dosing including drugs for local treatment of the respiratory system. Examples of liquid drugs that may be aerosolized include drugs in solution form, e.g., aqueous solutions, ethanol solutions, aqueous/ethanol mixture solutions, and the like, in colloidal suspension form, and the like. The invention may also find use in aerosolizing a variety of other types of liquids, such as insulin.

The aperture plates may be constructed of materials having a relatively high strength and that are resistive to corrosion. One particular material that provides such characteristics is a palladium nickel alloy. One particularly useful palladium nickel alloy comprises about 80% palladium and about 20% nickel. Other useful palladium nickel alloys are described generally in J. A. Abys, et al., “Annealing Behavior of Palladium-Nickel Alloy Electrodeposits,” Plating and Surface Finishing, August 1996, “PallaTech® Procedure for the Analysis of Additive IVS in PallaTech® Plating Solutions by HPLC” Technical Bulletin, Lucent Technologies, Oct. 1, 1996, and in U.S. Pat. No. 5,180,482, the complete disclosures of which are herein incorporated by reference.

Aperture plates constructed of such a palladium nickel alloy have significantly better corrosion resistance as compared to nickel aperture plates. As one example, a nickel aperture plate will typically corrode at a rate of about 1 micron per hour when an albuterol sulfate solution (PH 3.5) is flowing through the apertures. In contrast, the palladium nickel alloy does not experience any detectable corrosion after about 200 hours. Hence, the palladium nickel alloy may be used with a variety of liquids without significantly corroding the aperture plate. Examples of liquids that may be used and which will not significantly corrode such an aperture plate include albuterol, chromatin, and other inhalation solutions that are normally delivered by jet nebulizers, and the like.

While aperture plates can be constructed of a single metal, embodiments of the invention allow laminated metal layers to be utilized to form the body of the aperture plate. Thus, a base layer of material, e.g., nickel, could be utilized with a layer of corrosion resistive material such as a palladium nickel layer. The corrosion resistive material may be deposited only on areas that will contact the liquid up to the entire aperture plate. For example, the corrosive resistive material may be only within the apertures. Alternatively, additional areas, such as the bottom surface of the aperture plate may also be covered. In this fashion, a cheaper material can be utilized as a base while the often more expensive corrosion resistive layer can be utilized as a laminating layer over the base material. Thus, a corrosion resistive aperture plate body can be achieved in a less expensive fashion. Examples of other base materials include nickel alloys, nickel manganese, copper, tin and the like, and examples of other corrosion resistive materials include palladium, platinum, rhodium, palladium cobalt, gold and the like.

The apertures of the aperture plates will typically have an exit opening having a diameter in the range from about 1 micron to about 10 microns, to produce droplets that are about 2 microns to about 10 microns in size. In another aspect, the taper at the exit angle is preferably within the desired angle range for at least about the first 15 microns of the aperture plate. Beyond this point, the shape of the aperture is less critical. For example, the angle of taper may increase toward the opposite surface of the aperture plate.

Conveniently, the aperture plates of the invention may be formed in the shape of a dome as described generally in U.S. Pat. No. 5,758,637, previously incorporated by reference. Typically, the aperture plate will be vibrated at a frequency in the range from about 45 kHz to about 200 kHz when aerosolizing a liquid. Further, when aerosolizing a liquid, the liquid may be placed onto a rear surface of the aperture plate where the liquid adheres to the rear surface by surface tension forces. Upon vibration of the aperture plate, liquid droplets are ejected from the front surface as described generally in U.S. Pat. Nos. 5,164,740, 5,586,550 and 5,758,637, previously incorporated by reference.

The aperture plates of the invention may be constructed using an electrodeposition process where a metal is deposited from a solution onto a conductive mandrel by an electrolytic process. In one particular aspect, the aperture plates are formed using an electroforming process where the metal is electroplated onto a mandrel. When the desired thickness of deposited metal has been attained, the aperture plate is separated from the mandrel. Electroforming techniques are described generally in E. Paul DeGarmo, “Materials and Processes in Manufacturing” McMillan Publishing Co., Inc., New York, 5^{th} Edition, 1979, the complete disclosure of which is herein incorporated by reference.

The mandrels that may be utilized to produce the aperture plates of various embodiments of the invention may comprise a conductive surface having a plurality of spaced apart nonconductive islands. In this way, when the mandrel is placed into the solution and current is applied to the mandrel, the metal material in the solution is deposited onto the mandrel. Examples of metals which may be electrodeposited onto the mandrel to form the aperture plate have been described above.

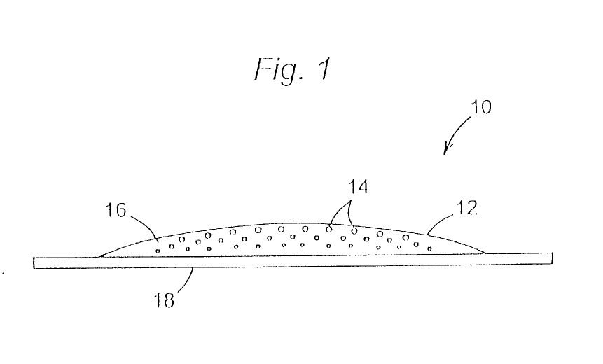

Referring now to FIG. 1, one embodiment of an aperture plate 10 will be described. Aperture plate 10 comprises a plate body 12 into which are formed a plurality of tapered apertures 14. Plate body 12 may be constructed of laminated metal layers, such as a palladium nickel alloy bonded over a nickel base, among others. Conveniently, plate body 12 may be configured to have a dome shape as described generally in U.S. Pat. No. 5,758,637, previously incorporated by reference. Plate body 12 includes a top or front surface 16 and a bottom or rear surface 18. In operation, liquid is supplied to rear surface 18 and liquid droplets are ejected from front surface 16.

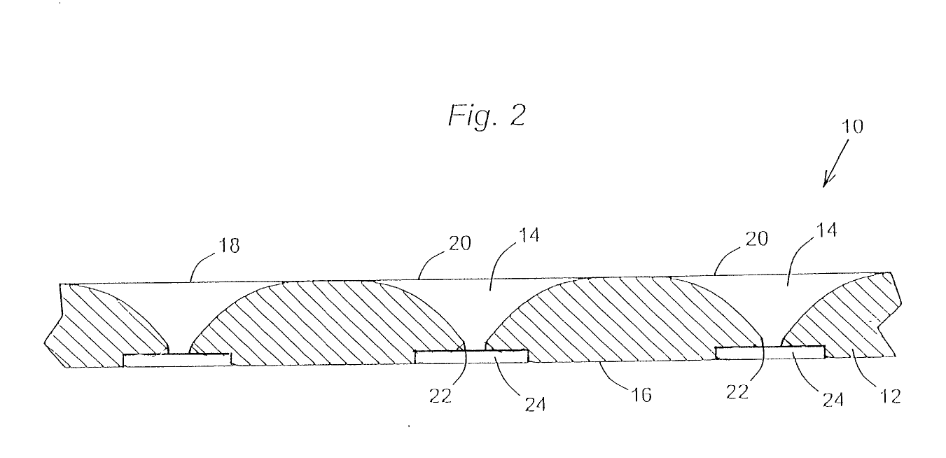

Referring now to FIG. 2, the configuration of apertures 14 will be described in greater detail. Apertures 14 are configured to taper from rear surface 18 to front surface 16. Each aperture 14 has an entrance opening 20 and an exit opening 22. With this configuration, liquid supplied to rear surface 18 proceeds through entrance opening 20 and exits through exit opening 22. As shown, plate body 12 further includes a recessed portion 24 adjacent exit opening 22. Recessed portion 24 is created from the manufacturing process employed to produce aperture plate 10 and may have a variety of shapes depending on the configuration of the mandrel.

In operation, liquid is applied to rear surface 18. Upon vibration of aperture plate 10, liquid droplets are ejected through exit opening 22. In this manner, the liquid droplets will be propelled from front surface 16. Although exit opening 22 is shown inset from front surface 16, it will be appreciated that other types of manufacturing processes may be employed to place exit opening 22 directly at front surface 16. It will be appreciated that the invention is not intended to be limited by this specific example. Further, the rate of production of liquid droplets may be varied by varying the exit angle, the exit diameter and the type of liquid being aerosolized. Hence, depending on the particular application (including the required droplet size), these variables may be altered to produce the desired aerosol at the desired rate.

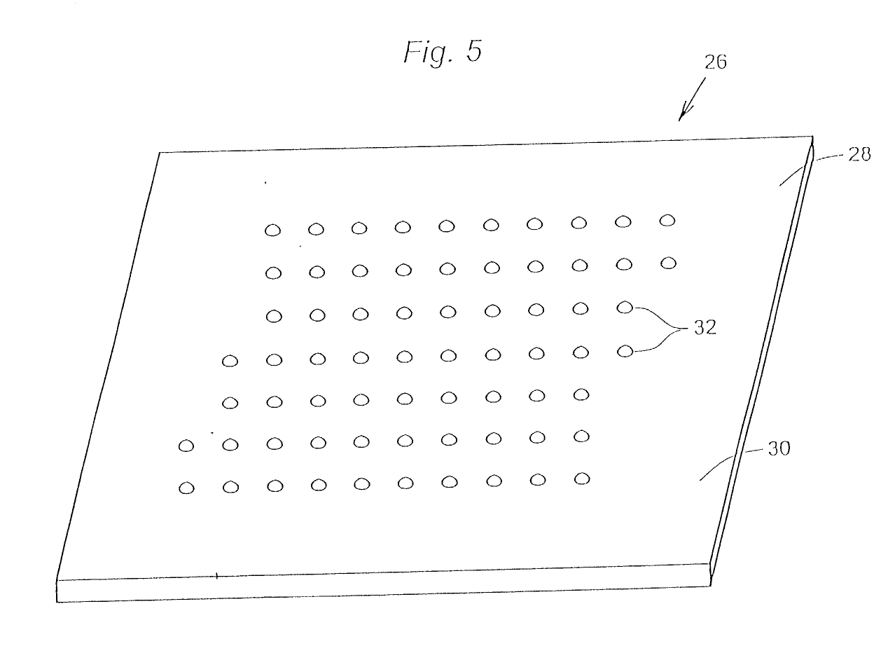

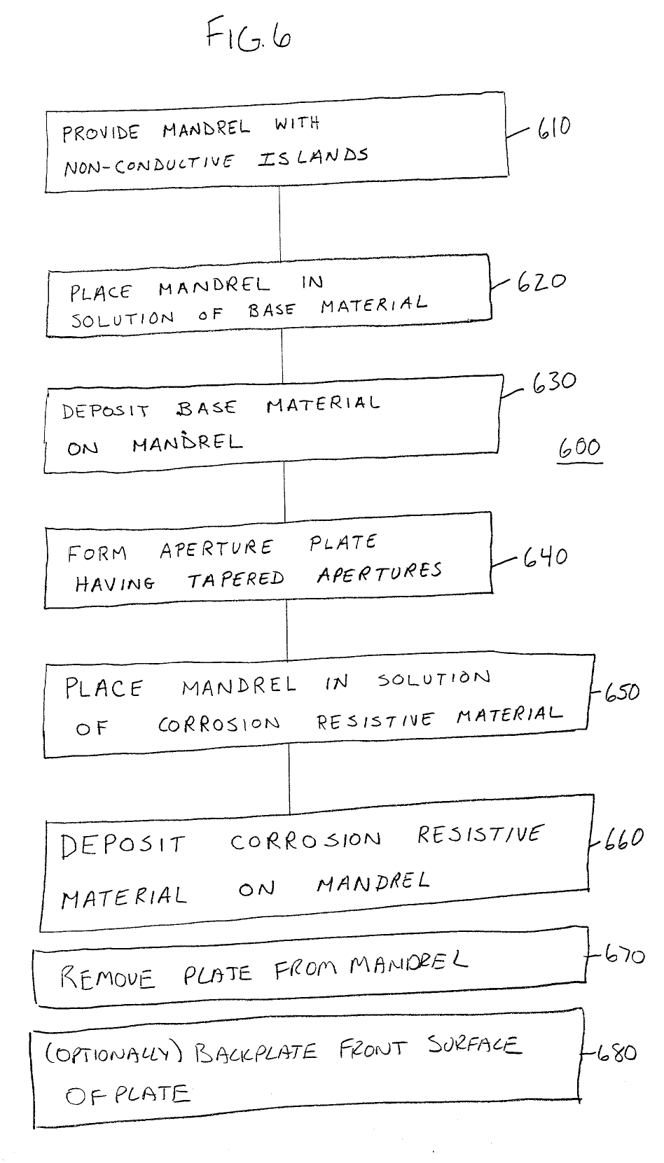

Referring now to FIGS. 3 and 3A, one embodiment of aperture plate 10 when laminated is shown. For convenience of illustration, in FIG. 3 a cross sectional view of only one aperture 14 in aperture plate 10 is shown. However, as previously described, aperture plate 10 is comprised of many of these apertures 14. In FIG. 3 a base material 34 is deposited, on a mandrel for example, and an initial aperture is formed. The base material 34 is typically a less expensive material than the eventual material which will be deposited above the base material; yet, the base material does not typically have the desired characteristic for interacting with a medication being dispensed. Therefore, a covering layer can be secured to the base material so as to facilitate dispensing of the medication without significant degradation of the aperture plate. Consequently, in FIG. 3, an outer material 36 is shown deposited on or bonded to the base material 34. For example, in one embodiment a base material of nickel can be deposited on a mandrel such as that shown in FIG. 5 to form aperture 14. Then, a second layer of metal, such as palladium or palladium nickel, can be deposited as layer 36 in FIGS. 3 and 3A.

Palladium nickel alloys possess the quality of having significant corrosion resistance as compared to nickel when dispensing typical medications. Therefore, they serve as a preferable material for use as a contact material for dispensing such medications.

However, nickel is a less expensive material and is utilized as shown in FIG. 3 to serve as a significant portion of the aperture plate. Examples of other base materials include nickel alloys, nickel manganese, copper, tin, and examples of other outer materials include palladium, platinum, rhodium, palladium cobalt, gold, and the like.

Once the initial layer 36 is deposited, yet another layer can be back plated onto the top surface of the aperture plate after the aperture plate has been removed from the mandrel. This is shown as layer 38 in FIGS. 3 and 3A. As can be seen in FIG. 3, the back plating material can penetrate the aperture 14 so as to cover a portion of the tapered walls.

Furthermore, the initial deposit of corrosion resistive material 36 is shown also covering the tapered walls and bottom surface of the aperture plate.

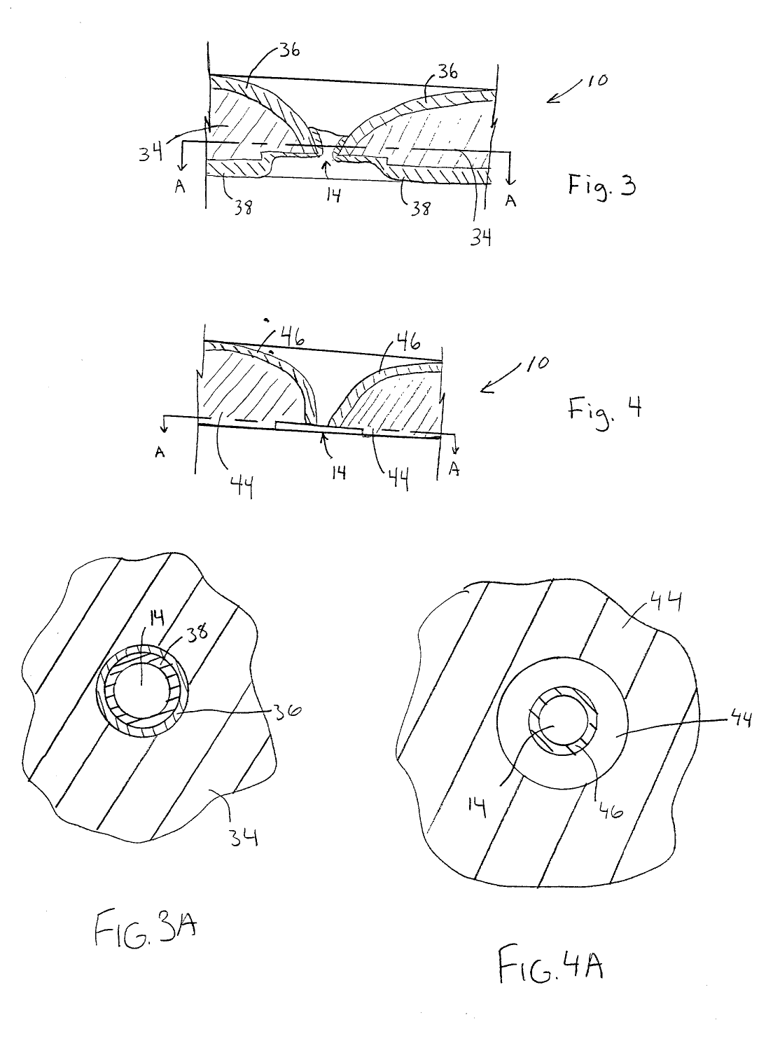

While the embodiment of FIG. 3 allows material to be deposited on both the bottom and top surfaces of the aperture plate, it is not necessarily required that the top surface be back plated with a corrosion resistive material. As shown in FIGS. 4 and 4A, an embodiment of the invention is provided in which the back or rear surface of aperture plate 10 is coated with a corrosion resistive material while the top surface is not treated in this way. Thus, in FIGS. 4 and 4A a base material 44 is deposited to serve as a base layer for the aperture plate 10. Furthermore, a second layer 46 is shown deposited on the base layer 44. Again, this can be accomplished by utilizing nickel as the base material 44 and utilizing palladium nickel as a corrosion resistive material for dispensing medications. The corrosion resistive material is shown in FIG. 4 coating both the bottom surface and tapered aperture.

While the percentage of the plate body made up of the base material can vary, in one embodiment the base material can make up more than 50% of the plate body, more preferably more than 75% of the plate body, and even more preferably more than about 95% of the plate body.

Referring now to FIG. 5, one embodiment of an electroforming mandrel 26 that may be employed to construct aperture plate 10 of FIG. 1 will be described. Mandrel 26 comprises a mandrel body 28 having a conductive surface 30. Conveniently, mandrel body 28 may be constructed of a metal, such as stainless steel. As shown, conductive surface 30 is flat in geometry. However, in some cases it will be appreciated that conductive surface 30 may be shaped depending on the desired shape of the resulting aperture plate.

Disposed on conductive surface 30 are a plurality of nonconductive islands 32. Islands 32 are configured to extend above conductive surface 30 so that they may be employed in electroforming apertures within the aperture plate. Islands 32 may be spaced apart by a distance corresponding to the desired spacing of the resulting apertures in the aperture plate. Similarly, the number of islands 32 may be varied depending on the particular need.

As shown, island 32 is generally conical or dome shaped in geometry.

Conveniently, island 32 may be defined in terms of a height h and a diameter D. As such, each island 32 may be said to include an average angle of incline or slope that is defined by the inverse tangent of ½ (D)/h. The average angle of incline may be varied to produce the desired exit angle in the aperture plate as previously described. Further, other shapes may be used as well. For example, the islands may be cylindrical in geometry.

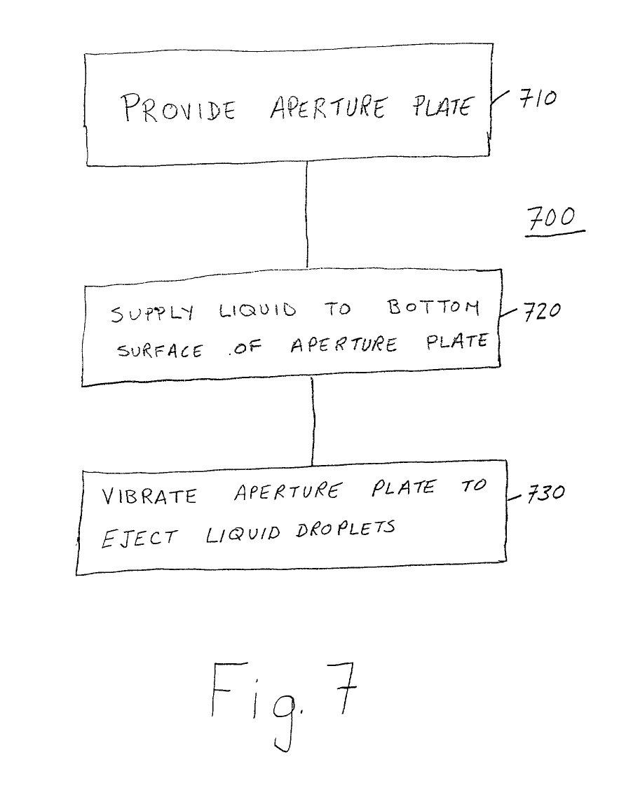

Referring now to FIG. 6, a method 600 is illustrated for manufacturing an aperture plate. In block 610 of FIG. 6, a mandrel (such as the mandrel in FIG. 5) is provided having non-conductive islands. The mandrel is placed in a solution of base material as shown in block 620. As noted earlier, one base material that could be used is nickel. Block 630 shows that the base material can then be deposited on the mandrel through a process such as electroplating. In electroplating, an electric current is supplied to the mandrel to deposit the material onto the mandrel so as to form an aperture plate. In one embodiment, the plating time for nickel lasts from 45-60 minutes.

To obtain the desired angle and the desired exit opening on the aperture plate 10, the time during which the electric current is supplied to the mandrel can be varied. Further, the type of solution into which the mandrel is immersed may also be varied. Still further, the shape and angle of the islands may be varied so as to vary the exit angle of the apertures.

Electroplating of the mandrel can be controlled so that a front surface of the aperture plate does not extend above the top of an island, such as islands 32 in FIG. 5. The amount of electroplating time may be controlled to control the height of the aperture plate. As such, the size of the exit openings may be controlled by varying the electroplating time. Once the desired height of the aperture plate is obtained, the electrical current can cease and the mandrel can be removed from the aperture plate. Thus, as illustrated by block 640 in FIG. 6, an aperture plate is formed having tapered apertures.

In block 650, a corrosion resistive material is applied to the aperture plate. This may be accomplished in a variety of ways. For example, an intermediate step between applying the base material to the mandrel and applying the corrosion resistive material can take place. As one example, the intermediate step may be the application of a chemical to the deposited base material to facilitate the bonding of the corrosion resistive material to the base material. For example, if nickel is used as the base material, a chemical can be applied to the deposited nickel so as to facilitate the bonding of the corrosion resistive material, such as palladium nickel, to the nickel base layer. For instance, a simple chemical rinse manipulation could be used, such as an activating process to facilitate bonding. Alternatively, a chemical deposition that would add material to the plating and enhance bonding, e.g., an activation and strike, such as a palladium nickel strike layer, a nickel strike layer, a palladium strike layer, a gold strike layer, or the like could be used.

In block 660, the corrosion resistive material is deposited so as to cover at least a portion of the base layer of material. This may be accomplished by placing the mandrel containing the aperture plate into a solution containing the corrosion resistive material and applying current to the mandrel. For example, the plating time for plating a nickel based aperture plate with palladium nickel is approximately about 15 minutes. While the method illustrated uses only a base material layer and a layer of corrosion resistive material, it is also envisioned that one or more intermediate layers of material could be deposited between the base layer and the ultimate outer layer, e.g., corrosion resistive material layer, of the aperture plate.

As shown in FIG. 3, in addition to coating the tapered surfaces of the apertures in the aperture plate, as well as the back surface of the aperture plate, the front surface may also be coated. For example, after electroplating the mandrel so as to deposit a base layer and then deposit a layer of non-corrosive material, the resulting aperture plate can be removed from the mandrel (see block 670) and back-plated so as to deposit material on the front surface of the aperture plate (see block 680). It is envisioned that the front surface will typically be covered with the same material used in covering the back surface and tapered surfaces. For example, when a corrosion resistive material is deposited on the tapered surfaces and the back surfaces, it is also envisioned that such material would be back-plated on the front surface. However, in some instances this will be unnecessary as the dispensing of medicine or other product will not contact the front surface of the aperture plate. Therefore, it will be unnecessary to back-plate the front surface of the aperture plate in such instances.

Once the process of electroplating the aperture plate is completed, the resulting aperture plate could be heat treated. For example, in one embodiment the aperture plate could be heated at about 400 degrees Celsius for approximately one minute.

Referring now to FIG. 7, a flow chart for a method 700 is illustrated. In block 710 an aperture plate is provided. Such an aperture plate can be of a type having a plate body with a top surface, a bottom surface, and tapered walls that form more than one aperture which taper from the bottom surface to the top surface. The plate body can be comprised of a base material and a corrosion resistive material plating at least the tapered walls. In block 720 a liquid is applied to the bottom surface of the aperture plate. The aperture plate can be vibrated so as to eject liquid droplets through the apertures of the aperture plate as illustrated in block 730.

The aperture plates described herein may be used in other applications as well. For example, the aperture plates may be used as a non-vibrating nozzle where liquid is forced through the apertures. As one example, the apertures may be used with ink jet printers that use thermal or piezoelectric energy to force the liquid through the nozzles. The aperture plates of such embodiments of the invention may be advantageous when used as non-vibrating nozzles with ink jet printers due to their non-corrosive construction and because the apertures have a low resistance to flow due to a relatively short-necked region.

The invention has now been described in detail for purposes of clarity in understanding. However, it will be appreciated that certain changes and modifications may be practiced within the scope of the appended claims.

What is claimed is:

1. An aperture plate comprising:
a plate body having a top surface, a bottom surface and tapered walls that form a plurality of apertures that taper from the bottom surface to the top surface, wherein the plate body comprises a base material and a corrosion resistive material plating at least the tapered walls.

2. An aperture plate as in claim 1, wherein the base material comprises at least about 50% of the plate body.

3. An aperture plate as in claim 1, wherein the base material is electroformed into a first layer and the corrosive resistive material is electroformed into a second layer on the bottom surface and the tapered walls.

4. An aperture plate as in claim 1, wherein the base material comprises nickel.

5. An aperture plate as in claim 1, wherein the corrosion resistive material comprises palladium nickel.

6. An aperture plate as in claim 1, wherein a portion of the plate body is dome shaped in geometry.

7. An aperture plate as in claim 1, further comprising a back plate material deposited on the top surface.

8. A method for forming an aperture plate, the method comprising:
providing a mandrel comprising a mandrel body having a conductive surface and a plurality of non-conductive islands on the conductive surface;
placing the mandrel into a solution containing a base material;
applying electrical energy to the mandrel to deposit the base material on the mandrel and form an aperture plate having a top surface adjacent the mandrel, a bottom surface and a plurality of tapered walls that define a plurality of tapered apertures;
placing the mandrel having the aperture plate in a solution containing a corrosion resistive material;
applying electrical energy to the mandrel to deposit the corrosion resistive material onto the bottom surface and the tapered walls.

9. A method as in claim 8, and further comprising:
removing the aperture plate from the mandrel.

10. A method as in claim 8 and further comprising back plating a material onto the top surface.

11. A method as in claim 8 and further comprising subjecting the base material to an intermediate step to facilitate bonding of the corrosion resistive material to the base material.

12. A method as in claim 8, wherein the intermediate step comprises exposing the base material to a chemical.

13. A method as in claim 8, further comprising heat treating the aperture plate.

14. The method as in claim 8, wherein the base material comprises nickel and the corrosion resistive material comprises palladium nickel.

15. The method as in claim 8, further comprising forming a dome in the plate body.

16. The method as in claim 8, wherein the base material comprises at least about 50% of the aperture plate.

17. A method for aerosolizing a liquid, the method comprising:
providing an aperture plate comprising a plate body having a top surface, a bottom surface and tapered walls that form a plurality of apertures that taper from the bottom surface to the top surface, wherein the plate body comprises a base material and a corrosion resistive material plating at least the tapered walls;
supplying a liquid to the bottom surface; and
vibrating the aperture plate to eject liquid droplets through the apertures.
